(19) United States
(12) Patent Application Publication
(10) Pub. No.: US 2018/0014957 A1
(43) Pub. Date: Jan. 18, 2018

(54) STENT RECONSTRAINMENT SYSTEM FOR MINIMAL VOLUME IN LOW PROFILE DELIVERY SYSTEM

(71) Applicant: BOSTON SCIENTIFIC SCIMED, INC., Maple Grove, MN (US)

(72) Inventors: Colby Harris, Weston, MA (US); Jonathan Root, Groveland, MA (US)

(21) Appl. No.: 15/647,694

(22) Filed: Jul. 12, 2017

Related U.S. Application Data
(60) Provisional application No. 62/361,585, filed on Jul. 13, 2016.

(51) Int. Cl. A61F 2/966; A61F 2/90

(52) U.S. Cl. CPC A61F 2/966; A61F 2/90; A61F 2220/0083; A61F 2002/9665

(57) ABSTRACT

A stent delivery device is provided that includes an outer tubular member, an intermediate tubular member disposed and slidable within a lumen of the outer tubular member, and a plurality of elongate members slidable within the intermediate tubular member. The plurality of elongate members have distal end structures that are moveable between a stent retaining configuration to selectively retain a stent and a stent releasing configuration to selectively release the stent.

### CROSS-REFERENCE TO RELATED APPLICATIONS

This application claims priority to U.S. Provisional Application No. 62/361,585, filed Jul. 13, 2016, the entire disclosure of which is herein incorporated by reference.

### TECHNICAL FIELD

The disclosure is directed to devices and methods for transporting, loading, and delivering stents. More particularly, the disclosure is directed to methods and systems for loading and delivering radially distensible stents using a retrieval suture.

### BACKGROUND

An intraluminary prosthesis is a medical device used in the treatment of diseased bodily lumens. One type of intraluminary prosthesis used in the repair and/or treatment of diseases in various body lumens is a stent. A stent is generally a longitudinal tubular device formed of biocompatible material which is useful to open and support various lumens in the body. For example, stents may be used in a bodily lumen, such as in the coronary or peripheral vasculature, esophagus, trachea, bronchi, colon, biliary tract, urinary tract, prostate, brain, as well as in a variety of other applications in the body. These devices are implanted within the body lumen to open and/or reinforce collapsing or partially occluded sections of the lumen.

Stents generally include an open flexible configuration. This configuration allows the stent to be inserted through curved body lumens. Furthermore, this configuration allows the stent to be configured in a radially compressed state for intraluminary catheter implantation. Once properly positioned adjacent the damaged treatment site in the body lumen, the stent is radially expanded so as to support and reinforce the body lumen. Radial expansion of the stent may be accomplished by inflation of a balloon attached to the catheter or the stent may be of the self-expanding variety which will radially expand once deployed and unconstrained. Tubular shaped structures, which have been used as intraluminary stents, have included helically wound coils which may have undulations or zig-zags therein, slotted stents, ring stents, braided stents and open mesh wire stents, to name a few. Super-elastic materials, as well as metallic and polymeric shape memory materials have also been used to form stents.

Although stent delivery systems are well-known in the art, the assembly of such delivery systems is often complicated. Additionally, contemporary Endoscopy practitioners increasingly use plastic self-expanding stents. Unlike most metallic self-expanding stents, the plastic stents have a tendency to permanently deform or lose some of their ability to self-expand when stored in a compressed state for a prolonged period of time. These stents are therefore preferably loaded into the stent delivery system shortly before being implanted in a patient. However, such loading often involves numerous steps and requires the use of multiple components (e.g., tools and fixtures) that are not part of the stent delivery system. Also, even with these added devices, the physician or user is often required to finish the loading process by pushing the stent into the delivery system by hand. Loading a stent in this way is therefore often difficult, time-consuming and has the potential to damage the stent. Accordingly, there is a need for simplified methods of on-site loading of a stent into stent delivery systems, while minimizing the risk of damaging the stent in the process.

Devices for retrieval and repositioning of self-expanding stents often involve a retrieval suture threaded through the proximal end of the stent. When a retrieval mechanism such as forceps pulls on the suture, the suture collapses the end of the stent in a uniform manner to aid in retrieval and repositioning of the stent. The forceps or retrieval mechanism is often a separate element from the delivery device, requiring two separate devices to be used for stent delivery and retrieval. Reconstrainment of the stent also often requires added structures within the stent or at the distal end of the inner and/or outer sheaths of the delivery device, adding volume and increasing the profile of the device. There is an ongoing need to provide alternative delivery and retrieval devices to simplify the delivery and retrieval process and reduce the volume and profile of the delivery device.

### BRIEF SUMMARY

This disclosure provides design, material, and use alternatives for medical devices, including delivery systems.

In a first example, stent delivery device comprises an outer tubular member having a distal end, a longitudinal length, and defining a lumen, an intermediate tubular member having a distal end and defining a lumen, the intermediate tubular member disposed and slidable within the outer tubular member lumen, and a plurality of elongate members slidable within one or more lumens of the intermediate tubular member, the elongate members having distal end structures moveable between a stent retaining configuration and a stent releasing configuration.

Alternatively or additionally, in another example, the distal end structures are in the stent retaining configuration when the distal end structures are extended distal of the distal end of the intermediate tubular member and in the stent releasing configuration when the distal end structures are retracted proximally within the intermediate tubular member.

Alternatively or additionally, in another example, the distal end structures are in the stent retaining configuration when the distal end structures are extended distal of the distal end of the intermediate tubular member and in the stent releasing configuration when the distal end structures are retracted proximally within the intermediate tubular member lumen.

Alternatively or additionally, in another example, the distal end structures are configured to retain a stent when the stent is in both a fully expanded and a constrained configuration.

Alternatively or additionally, in another example, the one or more lumens of the intermediate tubular member includes a plurality of channels extending longitudinally along the intermediate tubular member, wherein each of the plurality of elongate members extends through a separate channel.

Alternatively or additionally, in another example, the distal end structures include hooks configured to retain a suture connected to a proximal end of a stent.

Alternatively or additionally, in another example, the hooks are configured to straighten and release the suture as the elongate members are withdrawn proximally into the intermediate tubular member channels.

Alternatively or additionally, in another example, the hooks have a width measured transverse to a longitudinal axis of the elongate members, wherein the width of the hooks is greater than a diameter of the channels.

Alternatively or additionally, in another example, the intermediate tubular member includes a recess in an outer wall thereof adjacent the distal end, wherein the elongate members are configured to pass through the recess and through a suture loop disposed within the recess when the elongate members are extended to the distal end of the intermediate tubular member in the stent retaining configuration, the elongate members configured to be retracted proximal of the recess to release the suture loop in the stent releasing configuration.

Alternatively or additionally, in another example, the recess extends circumferentially around an outer surface of the intermediate tubular member.

Alternatively or additionally, in another example, the outer tubular member, intermediate tubular member, and elongate members are all independently moveable relative to one another in a longitudinal direction.

Alternatively or additionally, in another example, the outer tubular member has an outer tubular member handle at a proximal end thereof, the intermediate tubular member has an intermediate tubular member handle at a proximal end thereof, and proximal ends of the elongate members are fixed to an actuation handle.

Alternatively or additionally, in another example, the actuation handle fixed to the elongate members is disposed between the outer tubular member handle and the intermediate tubular member handle.

In another example, a stent delivery device comprises an outer tubular member having a distal end, a longitudinal length, and defining a lumen, an intermediate tubular member positioned in the outer tubular member lumen, the intermediate tubular member defining a lumen and having a distal end and a longitudinal length greater than the longitudinal length of the outer tubular member, a plurality of elongate members extending through the intermediate tubular member, the elongate members having proximal and distal ends, the distal ends configured to move between a stent retaining configuration and a stent releasing configuration, the outer tubular member, the intermediate tubular member, and the elongate members being independently movable in a longitudinal direction, the stent delivery device having a fully unconstrained loaded configuration, a fully constrained delivery configuration, and a stent deployment configuration, wherein in both the fully unconstrained loaded and the fully constrained delivery configurations the distal ends of the elongate members are in the stent retaining configuration, positioned distal of the distal end of the intermediate tubular member, and wherein in the stent deployment configuration the distal ends of the elongate members are in the stent releasing configuration, positioned proximal of the distal end of the intermediate tubular member.

Alternatively or additionally, in another example, the distal ends of the elongate members include hooks configured to retain a suture connected to a proximal end of a stent, the hooks configured to straighten as the elongate members are withdrawn proximally into the intermediate tubular member.

Alternatively or additionally, in another example, the intermediate tubular member includes a recess in an outer wall thereof adjacent the distal end, wherein the elongate members are configured to pass through the recess and through a suture loop disposed within the recess and connected to a proximal end of a stent when the elongate members are extended to the distal end of the intermediate tubular member in the stent retaining configuration, the elongate members configured to be retracted proximal of the recess to release the suture loop in the stent releasing configuration.

Alternatively or additionally, in another example, wherein in the fully unconstrained loaded and stent delivery configurations, the distal end of the outer tubular member is positioned axially aligned with or proximal of the distal end of the intermediate tubular member, and in the fully constrained delivery configuration the distal end of the outer tubular member is positioned distal of the distal end of the intermediate tubular member.

In another example, a method for delivering a self-expanding stent into a bodily lumen, comprises radially contracting a stent on a stent delivery device, the stent delivery device comprising an outer tubular member having a distal end, a longitudinal length, and defining a lumen, an intermediate tubular member having a distal end and defining a lumen, the intermediate tubular member disposed and slidable within the outer tubular member lumen, an inner member slidable within the intermediate tubular member lumen, a plurality of elongate members slidable within the intermediate tubular member, the elongate members having distal end structures moveable between a stent retaining configuration and a stent releasing configuration, a self-expanding stent in an expanded configuration disposed over the inner member, a proximal end of the stent positioned distal of the distal ends of the intermediate and outer tubular members, and a suture attached to the proximal end of the stent, the suture removably held by the distal end structures of the elongate members in the stent retaining configuration. Radially contracting the stent onto the stent delivery device includes sliding the outer tubular member distally over the stent, thereby contracting the stent within the outer tubular member. The method further comprises advancing the stent delivery device into the bodily lumen, sliding the outer tubular member proximally until the distal end of the outer tubular member is proximal of the stent, thereby allowing the stent to expand, and moving the distal end structures of the plurality of elongate members into the stent releasing configuration thereby releasing the stent.

Alternatively or additionally, in another example, the distal end structures are in the stent retaining configuration when the distal end structures are extended distal of the distal end of the intermediate tubular member and moving the distal end structures into the stent releasing configuration includes retracting the distal end structures proximally within the intermediate tubular member.

Alternatively or additionally, in another example, the distal end structures include hooks configured to retain the suture connected to the proximal end of the stent when the hooks are positioned distal of the distal end of the intermediate tubular member, the hooks configured to straighten and release the suture as the hooks are retracted proximally into the intermediate tubular member lumen.

Alternatively or additionally, in another example, the intermediate tubular member includes a plurality of channels extending longitudinally along the intermediate tubular member lumen, wherein each of the plurality of elongate member extends through a separate channel, wherein the hooks have a width measured transverse to a longitudinal axis of the elongate members, wherein the width of the hooks is greater than a diameter of the channels.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed in the present document.

FIG. 1 is a partial cross-sectional view of a stent delivery system in accordance with an embodiment of the disclosure, with a stent in a locked and fully unconstrained configuration;

FIG. 2A is a partial cross-sectional view of a portion of a stent delivery device with stent-retaining hooks in an advanced configuration;

FIG. 2B is a partial cross-sectional view of the device of FIG. 2A with stent-retaining hooks in a retracted configuration;

FIG. 3 is a partial cross-sectional view of the stent delivery system of FIG. 1, with the stent in a fully constrained configuration;

FIG. 4 is a partial cross-sectional view of the stent delivery system of FIG. 1 with the stent in an unlocked and unconstrained configuration;

FIG. 5A is a partial cross-sectional view of a stent delivery system in accordance with another embodiment of the disclosure, with a stent in a locked and unconstrained configuration; and

FIG. 5B is a partial cross-sectional view the stent delivery system of FIG. 5A with the stent in an unlocked and unconstrained configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification. Definitions of certain terms are provided below and shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure. The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications may be disclosed, one of skill in the art, incited by the present disclosure, would understand desired dimensions, ranges and/or values may deviate from those expressly disclosed.

As used in this specification and the appended claims, the singular forms “a,” “an,” and “the” include or otherwise refer to singular as well as plural referents, unless the content clearly dictates otherwise. As used in this specification and the appended claims, the term “or” is generally employed to include “and/or,” unless the content clearly dictates otherwise.

It is noted that references in the specification to “an embodiment”, “an example”, “some embodiments”, “some examples”, “another embodiment”, “another example” etc., indicate that the embodiment or example described may include one or more particular features, structures, and/or characteristics. However, such recitations do not necessarily mean that all embodiments or examples include the particular features, structures, and/or characteristics. Additionally, when particular features, structures, and/or characteristics are described in connection with one embodiment or example, it should be understood that such features, structures, and/or characteristics may also be used connection with other embodiments and examples whether or not explicitly described unless clearly stated to the contrary.

References herein to the term “distal” and variants thereof refer to a direction away from an operator of the subject devices, while references to the term “proximal” and variants thereof refer to a direction towards the operator of the subject devices. Accordingly, when the terms “distal” and “proximal” are used herein in the context of an assembly device that is being deployed within a body, such as a human body, by an operator, the term “distal” refers to a location within or near the body that is further within the body than a location that is “proximal” or closer to the operator.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

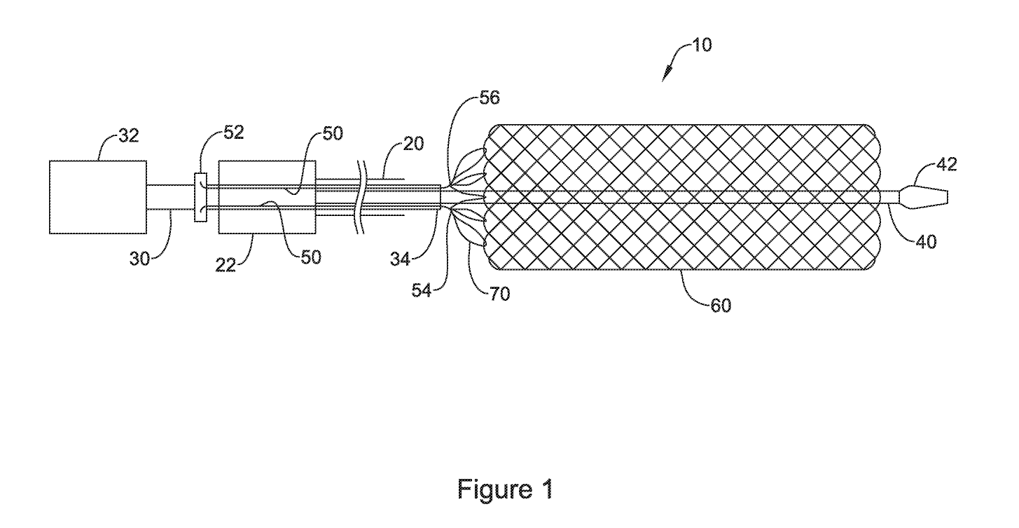

FIG. 1 is a cross-sectional view of a stent loading and delivery system or device 10 according to the present invention. The device 10 is particularly well suited for the loading, transluminal delivery and intraluminal deployment of a radially self-expanding prosthesis, such as a stent and/or a stent-graft, which may be radially compressed and loaded into device 10, transluminally delivered to an intended intraluminal treatment site, then released from the system for radial self-expansion against surrounding tissue. While the present device can be applied to the delivery of many intraluminary devices, it is particularly suited for delivering the self-expanding stent 60. The stent 60 is capable of being radially compressed and longitudinally extended for delivery into a bodily lumen. The degree of elongation depends upon the structure and materials of the stent 60 and may be quite varied. The diameter of the stent 60 also may become several times smaller as it elongates. The stent 60 may be constructed to self-expand when unconstrained, and thus released from a radially compressed state. Any stent that is capable of radial expansion may be used in accordance with the present device. For example, a radially distensible stent which does not substantially longitudinally elongate upon radial contraction is also useful. A non-limiting example of such a stent is one formed from zig-zag or undulating wires or wire. Thus, various stent types and stent constructions may be employed in the device 10, and the device 10 may be constructed to accommodate stents of various sizes and configurations.

One example of the present device applies to a braided stent 60. As used herein the term braiding and its variants refer to the diagonal intersection of elongate filaments so that each filament passes alternately over and under one or more of the other filaments, which is commonly referred to as an intersection repeat pattern. Useful braiding patterns include, but are not limited to, a diamond braid having a 1/1 intersection repeat pattern, a regular braid having a 2/2 intersection repeat pattern or a hercules braid having a 3/3 intersection repeat pattern. The passing of the filaments under and over one and the other results in slidable filament crossings that are not interlooped or otherwise mechanically engaged or constrained. Other examples of the stent 60, include other interwoven stent configurations, such as a knitted stent formed of a single filament.

While the stent 60 may be formed of metals, plastics or other materials, it is preferred that a biocompatible material or construction is employed. Useful biocompatible materials include, but are not limited to, biocompatible metals, biocompatible alloys, biocompatible polymeric materials, including synthetic biocompatible polymeric materials and bioabsorbable or biodegradable polymeric materials, materials made from or derived from natural sources and combinations thereof. Useful biocompatible metals or alloys include, but not limited to, nitinol, stainless steel, cobalt-based alloy such as Elgiloy, platinum, gold, titanium, tantalum, niobium, polymeric materials and combinations thereof. Useful synthetic biocompatible polymeric materials include, but are not limited to, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, silks and polytetrafluoroethylenes. The polymeric materials may further include a metallic, a glass, ceramic or carbon constituent or fiber. Useful and nonlimiting examples ofbioabsorbable or biodegradable polymeric materials include poly(L-lactide) (PLLA), poly(D,L-lactide) (PLA), poly(glycolide) (PGA), poly(L-lactide-co-D,L-lactide) (PLLAIPLA), poly(L-lactide-co-glycolide) (PLLAIPGA), poly(D,L-lactide-co-glycolide) (PLAIPGA), poly(glycolide-co-trimethylene carbonate) (PGAIPTMC), polydioxanone (PDS), Polycaprolactone (PCL), polyhydroxybutyrate (PHBT), poly(phosphazene) poly(D,L-lactide-co-caprolactone) PLAIPCL), poly(glycolide-co-caprolactone) (PGA/PCL), poly(phosphate ester) and the like. Further, the stent 60 may include materials made from or derived from natural sources, such as, but not limited to collagen, elastin, glycosaminoglycan, fibronectin and laminin, keratin, alginate, combinations thereof and the like.

Further, the stent 60 may be made from polymeric materials which may also include radiopaque materials, such as metallic-based powders or ceramic-based powders, particulates or pastes which may be incorporated into the polymeric material. For example, the radiopaque material may be blended with the polymer composition from which the polymeric wire is formed, and subsequently fashioned into the stent as described herein. Alternatively, the radiopaque material may be applied to the surface of the metal or polymer stent. Various radiopaque materials and their salts and derivatives may be used including, without limitation, bismuth, barium and its salts such as barium sulfate, tantalum, tungsten, gold, platinum and titanium, to name a few. Additional useful radiopaque materials may be found in U.S. Pat. No. 6,626,936, which is herein incorporated in its entirely by reference. Metallic complexes useful as radiopaque materials are also contemplated. The stent 60 may be selectively made radiopaque at desired areas along the stent or made be fully radiopaque, depending on the desired end-product and application. Further, portions of the stent 60, for example stent filaments, may have an inner core of tantalum, gold, platinum, iridium or combination of thereof and an outer member or layer of nitinol to provide a composite filament for improved radiocapacity or visibility. Alternatively, the stent 60 may also have improved external imaging under magnetic resonance imaging (MM) and/or ultrasonic visualization techniques. MM is produced by complex interactions of magnetic and radio frequency fields. Materials for enhancing Mill visibility include, but not be limited to, metal particles of gadolinium, iron, cobalt, nickel, dysprosium, dysprosium oxide, platinum, palladium, cobalt based alloys, iron based alloys, stainless steels, or other paramagnetic or ferromagnetic metals, gadolinium salts, gadolinium complexes, gadopentetate dimeglumine, compounds of copper, nickel, manganese, chromium, dysprosium and gadolinium. To enhance the visibility under ultrasonic visualization the stent 60 may include ultrasound resonant material, such as but not limited to gold. Other features, which may be included with the stent 60 of the present device, include radiopaque markers; surface modification for ultrasound, cell growth or therapeutic agent delivery; varying stiffness of the stent or stent components; varying geometry, such as tapering, flaring, bifurcation and the like; varying material; varying geometry of stent components, for example tapered stent filaments; and the like.

Also, the stent 60 may have coverings, films, coatings, and the like disposed over, under or throughout or embedding the stent 60. For example, the stent 60 may include a covering, such as a polymeric covering, disposed over the longitudinal length or a portion of the longitudinal length of the stent 60. Further, the stent 60 may include a liner, such as a polymeric liner, disposed within the longitudinal length or a portion of the longitudinal length of the stent 60. Moreover, the stent 60 may include both a covering and a liner, such as a polymeric covering and liner which include the same or different polymeric materials, disposed over and within the longitudinal length or a portion of the longitudinal length of the stent 60. The covering and/or the liner may be a unitary film or coating that embeds or partially embeds the stent 60. In some instances, the covering and/or liner may be applied to the stent 60 in a dip coating or spray coating process The covering and/or the liner may be in the form of a tubular structure, for example composed of polymeric material and/or silicone. The covering and/or the liner may also comprise any plastic or polymeric material, such as a somewhat hard but flexible plastic or polymeric material. The covering and/or the liner may be transparent or translucent, desirably substantially or partially transparent. Furthermore, the covering and/or the liner may be constructed of any suitable biocompatible materials, such as, but not limited to, polymers and polymeric materials, including fillers such as metals, carbon fibers, glass fibers or ceramics. Useful covering and/or the liner materials include, but are not limited, polyethylene, polypropylene, polyvinyl chloride, polytetrafluoroethylene (PTFE), including expanded polytetrafluoroethylene (ePTFE), fluorinated ethylene propylene, fluorinated ethylene propylene, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polyimides, polycarbonates, polyaldehydes, polyether ether ketone, natural rubbers, polyester copolymers, styrene-butadiene copolymers, polyethers, such as fully or partially halogenated polyethers, silicones, and copolymers and combinations thereof. The coating or coatings may be on the stent 60, components of the stent 60, and combinations thereof. The stent components, in part or in total, may be temporary, for example bioabsorbable, biodegradable, and the like, or may be permanent (i.e., not substantially bioabsorbable or biodegradable), for example the above-described biocompatible metals, alloys and polymers.

In some examples, the stent 60 may include braided polyester filaments, such as PET polyester filaments. Further, in some applications, the stent 60 may be embedded in a coating of silicone. The stent may be treated with a therapeutic agent or agents, such as, but not limited to, anti-thrombogenic agents (such as heparin, heparin derivatives, urokinase, and PPack (dextrophenylalanine proline arginine chloromethylketone); antiproliferative agents (such as enoxaprin, angiopeptin, or monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-miotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

Further, as depicted in FIG. 1, the stent 60 may have a straight or substantially straight longitudinal configuration. The present disclosure, however, is not so limited. For example, the stent 60 may have a varied diameter, such as a flaring or tapering, along a portion or portions of its longitudinal expanse. One non-limiting example of a varied diameter stent is a stent having a longitudinal body and a flared end at the first and/or second end of the stent (not shown). Flared ends may have a diameter greater than the diameter of the longitudinal body of the stent 60. The stent 60, however, is not so limited, and for example flared ends, individually or in combination, may have a smaller diameter that the diameter of at least a portion of the longitudinal body of the stent 60. Further, the stent 60 may be repositionable, removable and/or reconstrainable, and/or may include multiple interconnected or non-interconnected stents.

As shown in FIG. 1, the device 10 may include an elongate, flexible inner member 40 with a distal tip 42, disposed and slidable within the lumen of an intermediate tubular member 30, which is disposed and slidable within the lumen of an outer tubular member 20, interrelated as shown. The distal tip 42 is useful for navigating bodily lumens without causing trauma to the same. The outer tubular member 20 is sized to slide over, or otherwise be positioned over and surround the constrained stent 60, and thus constrain or radially compress the stent 60 in a radially constrained configuration, with the inner member 40 extending through the lumen of the stent 60. The intermediate tubular member 30 may remain proximal of the stent 60 throughout loading, delivering, and repositioning of the stent 60 with the device 10. An outer tubular member handle 22, such as a distal handle, may be disposed at and fixedly secured to the proximal end of the outer tubular member 20. An intermediate tubular member handle 32, such as a proximal handle, may be disposed at and fixedly secured to the proximal end of the intermediate tubular member 30. The inner member 40 may extend through the handle 32 and may have a separate handle (not shown) located proximal of the handle 32. A constrainment element 50, or a plurality of constrainment elements 50 may extend through and be slidable within a lumen or lumens of the intermediate tubular member 30 and extend out the distal end 34 of the intermediate tubular member 30. For example, two or more elongate constrainment elements 50 may extend through and are slidable within a lumen or lumens of the intermediate tubular member 30 and extend out the distal end 34 of the intermediate tubular member 30. In some instances the constrainment element(s) 50 may extend through the central lumen of the intermediate tubular member 30 along with and exterior of the inner member 40. In other instances, the intermediate tubular member 30 may include one or more additional lumens for receiving the constrainment element(s) 50 individually or collectively, while the inner member 40 extends through the central lumen of the intermediate tubular member 30. A constrainment element handle 52 (e.g., an actuation handle) may be fixed to the proximal end(s) of the constrainment element(s) 50. Movement of the handle 52 relative to the intermediate tubular member 30 may longitudinally move the constrainment elements 50 relative to the intermediate tubular member 30. The distal end 54 of each constrainment element 50 may have a distal end structure configured to be movable between a stent retaining configuration and a stent releasing configuration. The distal end structures may include hooks 56 configured to retain one or more loops of a suture 70 attached to the proximal end of the stent 60. In other instances, the distal end structure of the constrainment element(s) 50 may have a coil or other engagement structure to retain the suture 70.

The suture 70 may be threaded, braided, woven, knitted, or otherwise intertwined directly into the wires, filaments, or structure of the stent 60 itself. The suture 70 may be a separate element attached to the stent 60 or it may be braided or woven into the stent to form an integral part of the stent 60. In some instances, suture 70 may be a loop extending circumferentially around the end of the stent 60 passing in and out of end loops formed in the end of the stent 60 from the filaments of the mesh body of the stent 60. In other instances, suture 70 may be a monolithic extension of one or more filaments forming the mesh body of the stent 60 extending out of the mesh body. In some embodiments, loops formed at the proximal end of the stent 60 (not shown) may be captured by the hooks 56 instead of a separate suture element. It should be noted that references herein to the term “suture” denotes a length of thread, thread-like member, cord, filament, wire or other similar structure. It should be understood that sutures as referred to herein can be made of a single material or composite materials. Accordingly, the terms “suture,” “thread,” “cord,” “filament,” and/or “wire” are used interchangeably herein.

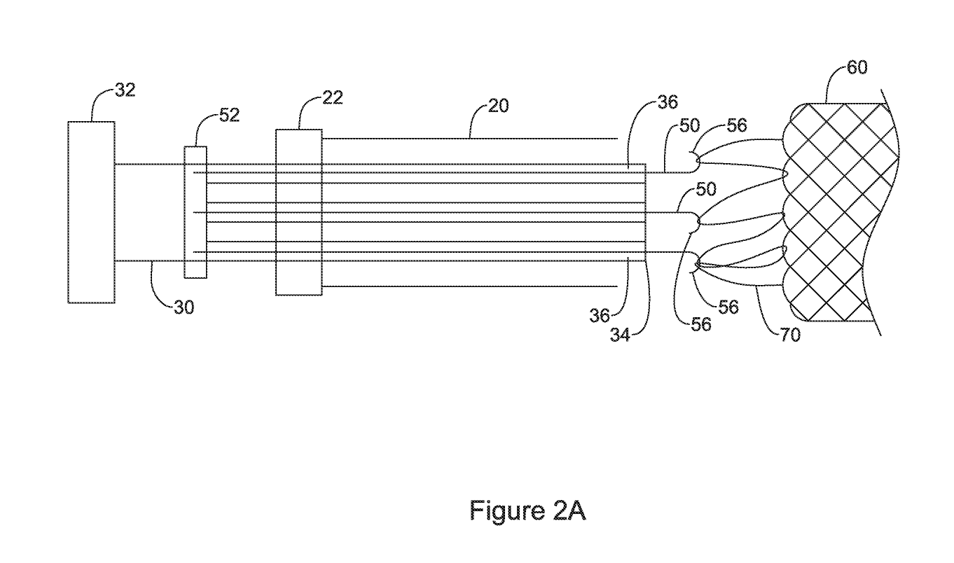

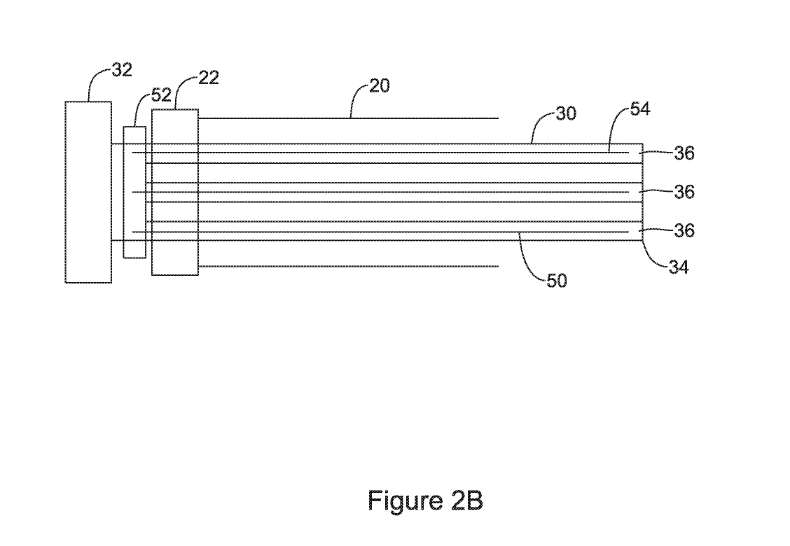

FIG. 2A is a partial cross-sectional view of the outer tubular member 20, the intermediate tubular member 30, the constrainment elements 50, stent 60, and suture 70, with the inner member 40 removed for clarity. The hooks 56 at the distal end of the constrainment elements 50 may be configured to move from their hook configuration, as seen in FIG. 2A, when the hooks are disposed distal of the distal end 34 of the intermediate tubular member 30, to a substantially straight, retracted configuration, as seen in FIG. 2B, when the distal ends 54 of the constrainment elements 50 are retracted into the lumen(s) of the intermediate tubular member 30. The constrainment elements 50 may be made of a material that allows for the distal ends 54 to be formed into hooks 56 with sufficient rigidity to retain suture loops during loading and radial contraction of the stent 60, but also allows for the hooks 56 to be straightened when the hooks 56 are retracted into the intermediate tubular member 30. The constrainment elements 50 may be made of monofilament wire, nitinol wire, Elgiloy wire, stainless steel wire, or any other wire that has the appropriate tensile strength to retain the suture loops while still allowing for straightening upon retraction into the intermediate tubular member 30. In some examples, the constrainment elements 50 may be made of a shape memory material, such as nitinol. The entire constrainment element 50, including the hook 56 may be made of the same material or the proximal elongate region of the constrainment elements 50 may be wires made of a first material with the distal region including the hooks 56 made of a second material fixed to the proximal region.

As seen in FIGS. 2A and 2B, the intermediate tubular member 30 may include an individual channel 36 through which each individual constrainment element 50 may pass through. For example, the intermediate tubular member 30 may include a plurality of individual channels 36, one for each of a plurality of constrainment elements 50. The channels 36 may be grooves, lumens or tubular structures extending along or through the wall of the intermediate tubular member 30. The channels 36 may extend the entire length of the intermediate tubular member 30 from the proximal end of the intermediate tubular member 30 to the distal end of the intermediate tubular member 30 or the channels may extend only through the distal region of the intermediate tubular member 30. The channels 36 may open out at a distally facing surface of the intermediate tubular member 30 at the distal end of the intermediate tubular member 30. The hooks 56 have a width measured transverse to a longitudinal axis of the elongated proximal portion of the constrainment elements 50, and the width of the hooks 56 may be greater than a diameter of the channels 36, so that the channels 36 may aid in straightening the hooks 56 upon retraction of the hooks 56 proximally into the channels 36. For example, proximal retraction of the constrainment elements 50 may bring the hooks 56 into engagement with the distal end of the intermediate tubular member 30, and further proximal retraction of the constrainment elements 50 with the hooks 56 contacting the distal end of the intermediate tubular member 30 will cause the hooks 56 to uncurl and straighten as the distal region of the constrainment elements including the hooks 56 are pulled into the channel 36 proximal of the distal end of the intermediate tubular member 30.

Manipulation or axial movement of the handles 22, 32, 52 permits independent axial movement of the outer tubular member 20, intermediate tubular member 30, and the constrainment elements 50, respectively. For example, the outer tubular member handle 22 may be axially moved between distal and proximal positions to so axially move the outer tubular member 20. Such movement may be done while keeping the other handles 32, 52 fixed or relatively fixed to allow independent or substantially independent movement of the outer tubular member 20 while the inner member 40, constrainment elements 50, and the intermediate tubular member 30 remain fixed or relatively fixed. This may allow the outer tubular member 20 to be moved distally over the stent 60 to constrain or radially compress the stent 60, and to be moved proximally to uncover and unconstrain the stent 60 and allow the stent 60 to expand, all the while maintaining the hooks 56 secured to the suture loops 70, due to the fixed positions of the intermediate tubular member 30 and the constrainment elements 50. In a similar fashion, the constrainment element handle 52 may be axially moved between distal and proximal positions to axially move the constrainment elements 50 within the intermediate tubular member 30 while keeping the other handles 22, 32 fixed or relatively fixed to allow independent or substantially independent movement of the constrainment elements 50 while the intermediate tubular member 30 and the outer tubular member 20 remain fixed or relatively fixed. This may allow the constrainment elements 50 to be moved from the stent retaining configuration with the hooks 56 disposed distal of the intermediate tubular member 30 in a hooked or curled configuration to the stent releasing configuration with the hooks 56 retracted proximally into the intermediate tubular member 30 with the hooks 56 in an uncurled or straightened configuration. Further, the handles 22 and 52 may be moved or manipulated in concert as a pair while keeping the third or non-paired handle 32 fixed or relatively fixed to allow concurrent movement of the constrainment elements 50 and the outer tubular member 20 while keeping the intermediate tubular member 30 fixed or relatively fixed. This may allow for the stent 60 to be uncovered by the outer tubular member 20, expanding the stent 60, followed closely by release of the stent from the hooks 56.

FIG. 1 depicts the stent 60 in a loading and transport position for the device 10. The handles 22 and 32 may be disposed relatively away from one another such that the hooks 56 on the distal ends 54 of the constrainment elements 50 are disposed distal of the distal end 34 of the intermediate tubular member 30. In such a configuration the handles 22 and 52 may be disposed relatively toward one another. The stent 60 may be placed over the inner member 40 with the distal end of the stent 60 proximal of the distal tip 42 and the proximal end of the stent 60 distal of the outer tubular member 20 and distal of the intermediate tubular member 30. A suture 70 may be threaded or woven through the proximal end of the stent 60. The suture 70 may be looped over the hooks 56 to secure or lock the stent onto the device 10. In other words, the hooks 56 may grab the suture 70. The stent 60 remains in a fully unconstrained configuration, however the hooks 56 retain the suture 70 thereby keeping the stent 60 attached to the delivery device 10. The distal end of the outer tubular member 20 remains proximal of the stent, allowing the stent 60 to remain in the fully unconstrained configuration for loading, packaging, and shipping.

Prior to delivery within a patient, the stent 60 may be constrained or radially contacted onto or about the inner member 40 by the outer tubular member 20. During the contraction of the stent 60, the proximal handle 32 and constrainment element handle 52 may be kept in relative constant axial positions relative to the outer tubular member handle 22. As such, the inner member 40, intermediate tubular member 30, and the constrainment elements 50 are also kept in relative constant axial positions with the distal end of the intermediate tubular member 30 being disposed proximal of the stent 60. The outer tubular member handle 22 may be moved distally away from the intermediate tubular member handle 32 and the constrainment element handle 52, thereby moving the outer tubular member 20 distally over the stent 60, compressing the stent 60 as the stent 60 is held in place by the constrainment element hooks 56, to complete the loading of the stent into the outer tubular member 20 prior to implantation of the stent 60 into a body lumen during a medical procedure. The hooks 56 may retain the suture 70 and thus the proximal end of the stent 60 in place during the constrainment process.

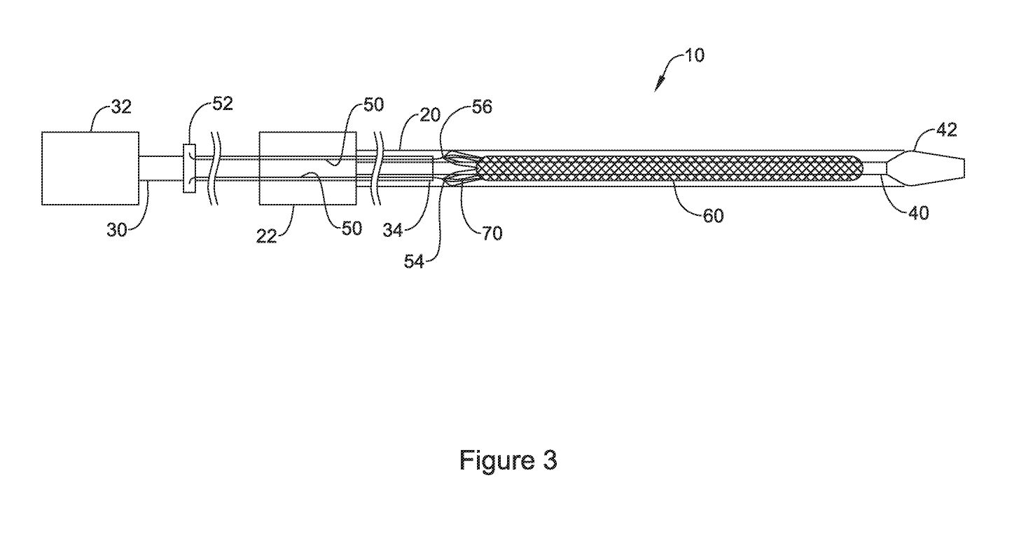

When fully constrained within the outer tubular member 20, the stent 60 is disposed within the outer tubular element 20 in a radially contracted configuration, as seen in FIG. 3. The hooks 56 and distal end of intermediate tubular member 30 remain proximal of the stent 60, leaving only the inner member 40 extending through the interior of the stent 60, and only the outer tubular element 20 disposed over and surrounding the stent 60. The hooks 56 may remain latched onto the suture 70 (e.g., hooked around the suture 70). As described above, the distal handle 22 may be pulled away axially distally from the proximal handle 32 and the constrainment element handle 52 in the stent loaded position as compared the handle 22, 32, 52 positions of FIG. 1. In other words, the outer tubular member 20 may be advanced distally toward the distal tip 42 to cover the stent 60 in the fully constrained configuration. The hooks 56 releasably secure the stent 60 within the outer tubular member 20. The stent 60 may be compressed directly onto the inner member 20 with an inner surface of the stent 60 along its entire length facing and/or contacting the peripheral surface of the inner member 40. In the example shown in FIG. 3, no structures are present between the inner member 40 and the stent 60, or between the stent 60 and the outer tubular member 20, thus the device 10 may be free or substantially free of tubes, bands, barbs, pins or protrusions which may engage and possibly damage the stent 60 during delivery.

This delivery device configuration provides the advantages of reducing bulk and providing a low profile delivery system due to the absence of additional material especially in the proximal region of the stent in the form of a compression sleeve, stent holding member, or reconstrainment band. The only structure inside the stent 60 may be the inner member 40 and the only structure on the outer surface of the stent 60 may be the outer tubular member 20. This may reduce the pressure on the stent 60, which may reduce the rate of auto-adhesion, as well as reducing the necessary deployment forces for deploying the stent 10 from the delivery device 10.

When the device 10 is at the desired location with a body lumen (not shown) and the stent 60 is ready to be deployed, the distal handle 22, and thus the outer tubular member 20, may be moved proximally, thereby moving the outer tubular member 20 axially away from the distal tip 42 and releasing the stent 60. The distal handle 22 may be moved proximally until just before it contacts the constrainment element handle 52, for example. At this point, the outer tubular member 20 may be disposed entirely proximal of the proximal end of the stent 60 and/or the distal end 34 of the intermediate tubular member 30, allowing the stent 60 to fully expand. This configuration is seen in FIG. 1. However, even once the stent 60 is fully unconstrained by the outer tubular member 30 and radially expanded in the body lumen, the hooks 56 retain the stent 60 in a loaded or captured configuration, preventing premature release of the stent 60 from the delivery device 10. In the event it is desired to re-constrain the stent 60 into the outer tubular member 20, such as to reposition the stent 60, the stent 60 may be re-loaded into the outer tubular member 20.

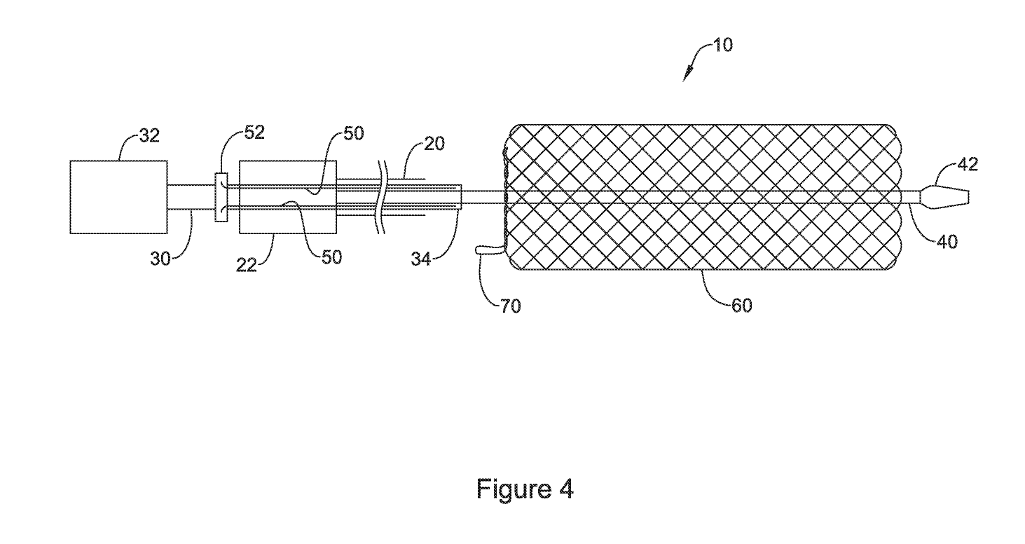

The stent 60 may be released from the delivery device 10 by moving the constrainment element handle 52 proximally to actuate the constrainment elements proximally. For example, the user may continue to move the distal handle 22 in a proximal direction, until it contacts the constrainment element handle 52. Further proximal movement of the distal handle 22 also moves the constrainment element handle 52 in the proximal direction, which pulls the constrainment elements 50 proximally through the intermediate tubular member channels 36. When the hooks 56 reach the distal end 34 of the intermediate tubular member 30, the hooks 56 are uncurled or straightened as they enter the intermediate tubular member, and the straightened hooks 56 release the suture 70, thereby fully releasing the stent 60, as shown in FIG. 4.

Alternatively, once the distal handle 22 has moved the outer tubular member 20 proximal of the stent 60, the constrainment element handle 52 may be moved alone in a proximal direction in order to retract the constrainment elements 50 into the intermediate tubular member to uncurl or straighten the hooks 56 and thereby release the suture 70 and the stent 60. The hooks 56 at the distal ends 54 of the constrainment elements 50 may be uncurled or straightened as they enter one or more lumens of the intermediate tubular member. In embodiments with channels 36, the hooks 56 are uncurled or straightened as they enter the channels 36, as shown in the difference between FIGS. 2A and 2B. Once the stent 60 is released from the constrainment elements 50, the delivery device 10 may be removed completely by moving the device 10 in the proximal direction, withdrawing the inner member 40 and distal tip 42 through the expanded stent 60.

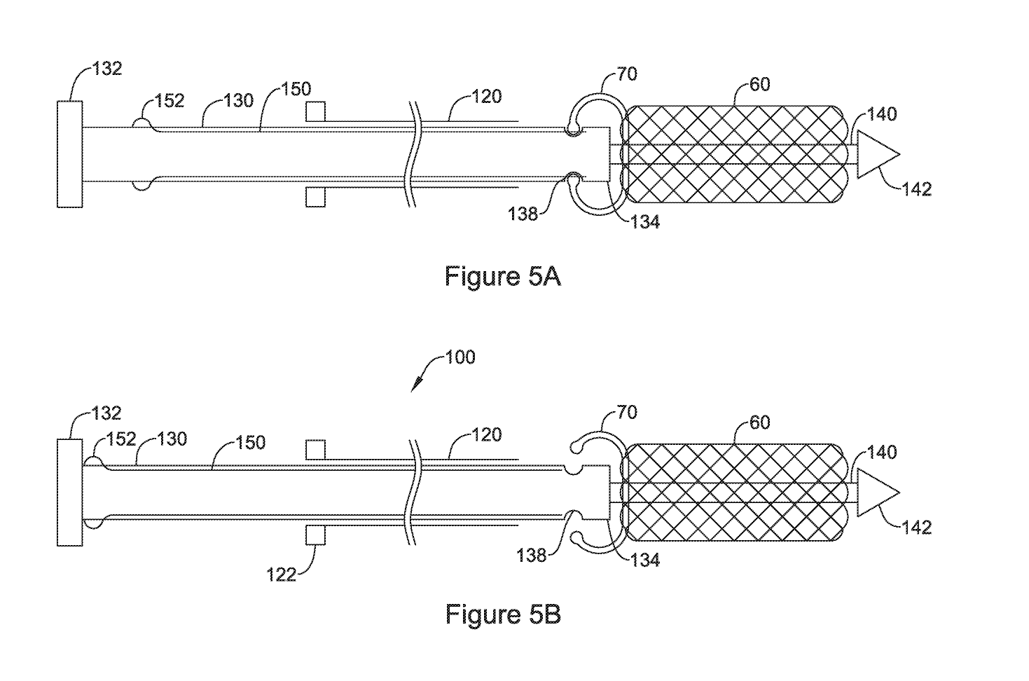

Another example of a stent delivery and deployment device 100 is shown in FIGS. 5A and 5B. The device 100 may include an elongate, flexible inner member 140 with a distal tip 142, disposed slidably within the lumen of an intermediate tubular member 130, which is disposed slidably within the lumen of an outer tubular member 120, interrelated as shown. The outer tubular member 120 may be sized to slide over and surround the stent 60, thus radially constraining the stent 60, which is disposed over the inner member 140. The intermediate tubular member 130 may remain proximal of the stent 160 throughout loading, delivering, and repositioning of the stent 60 with the device 100. An outer tubular member handle 122 may be disposed at and fixedly secured to the proximal end of the outer tubular member 120. An intermediate tubular member handle 132 may be disposed at and fixedly secured to the proximal end of the intermediate tubular member 130. The inner member 140 may extend through the handle 132 and may have a separate handle (not shown) located proximal of the handle 132. A constrainment element 150, or a plurality of constrainment elements 150 may extend through and be slidable within a lumen or lumens of the intermediate tubular member 130 and extend out the distal end 134 of the intermediate tubular member 130. For example, a plurality of constrainment elements 150 may extend through one or more lumens of the intermediate tubular member 130 to a position adjacent or distal of the distal end 134. The proximal ends of the constrainment elements 150 may be fixed to a constrainment element handle 152 (i.e., an actuation handle) disposed between the distal handle 122 and the proximal handle 132. The intermediate tubular member 130 may have a groove or recess 138 in an outer wall thereof, adjacent the distal end 134. To lock the stent 60 to the delivery device 100, a suture 70 may be attached to the proximal end of the stent 60. Loops of the suture 70 may be placed into the recess 138 and the constrainment elements 150 may be advanced distally until the constrainment elements 150 pass through the recess 138 and through the suture loop 70, trapping the suture 70 within the recess 138. The constrainment elements 150 may be substantially rigid elements which, when traversing the recess 138, prevent the suture 70 from being removed from the recess 138. The constrainment elements 150 may extend through channels 36 within the lumen of the intermediate tubular member 130, as shown in FIG. 2A. In other instances, the channels 36 may be separate channels extending through the intermediate tubular member 130, separate from the lumen through which the inner member 140 extends. The channels 36 may extend across the recess 138. The suture 70 may be locked onto the delivery device 100 by placing a loop of the suture 70 into the recess 138 on opposite sides of the intermediate tubular member 130 when two constrainment elements 150 are present. As the handle 152 is moved distally the constrainment elements 150 cross the recess 138 and capture the loops of the suture 70 within the recess 138. The recess 138 may extend circumferentially around the outer wall of the intermediate tubular member 130, as shown in FIGS. 5A and 5B, or individual, discontinuous recesses may be present that line up with the individual constrainment elements 150.

With the stent 60 locked to the device 100, the assembly may be packaged and shipped in the configuration of FIG. 5A, with the stent fully expanded. Prior to use, the stent 60 may be radially constrained within the outer tubular member 120 as in the example described above and shown in FIG. 3. The outer tubular member 120 may be advanced distally to constrain the stent 60 therein. Once the stent 60 is delivered to the desired location within the body of the patient, the stent 60 may be radially expanded and deployed by moving the outer tubular member 120 proximally until the entire outer tubular member 120 is disposed proximal of the proximal end of the stent 60 and/or the distal end 134 of the intermediate tubular member 130. The constrainment handle 152 may then be moved proximally to withdraw the constrainment elements 150 proximally of the recess 138, thereby releasing the suture 70 from the recess 138, as shown in FIG. 5B. Once the stent 60 is fully expanded and released from the constrainment elements 150, the device 100 may be removed proximally, leaving the stent 60 in place within the body lumen.

The above described features provide, among other things, reconstrainability of the stent 60 within the system or device 10, to allow the stent 60 to be moved within the bodily lumen after initial placement but before the stent is released from the device 10. For example, prior to delivery, with the suture 70 attached to the hooks 56 or other constrainment elements, the outer tubular member 20 may be advanced distally over the stent 60 to radially compress the stent 60 relative to the position of the inner member 40. The device 10 may then be delivered to a first location within the bodily lumen. The outer tubular member 20 may be retracted proximally past the proximal end of the stent 60 and/or the distal end 34 of the intermediate tubular member 30, until the distal handle 22 is just short of the constrainment element handle 52, allowing the stent 60 to radially expand while remaining coupled or tethered to the device 10 via the hooks 56 (or other constrainment elements) and suture 70. If it is determined that the stent 60 is not in the desired location, the stent 60 may be reconstrained by moving the outer tubular member 20 distally to reconstrain the stent 60 at least partially within the outer tubular member 20, while maintaining the position of the hooks 56 (or other constrainment elements) distal of the distal end 34 of the intermediate tubular member 30, thereby keeping the stent 60 coupled or tethered onto the device 10. After the stent 60 is repositioned, the stent 60 may again be allowed to expand by moving the outer tubular member 20 proximally to uncover, and thus unconstrain the stent 60. When the stent 60 is in the desired position, the distal handle 22 and/or constrainment element handle 52 are moved proximally to withdraw the constrainment elements and hooks 56 into the intermediate tubular member 30, thereby uncurling or straightening the hooks 56 to release the suture 70, as shown in FIG. 4.

The structural features of the device discussed above may suitably be combined in any combination according the present invention. In other words, all possible combinations of the features or structural elements of the present invention are contemplated, including all features and structural elements described in conjunction with the drawings.

Use of the device 10, 100 is also contemplated by the present disclosure. Use of the device 10, 100 may include a method for loading a self-expanding stent 60 into a delivery and deployment device 10, 100 and delivering the stent 60 to a bodily lumen. The method may include radially contracting a stent 60 on a delivery device 10, 100, the device 10, 100 including an outer tubular member 20, 120 having opposed proximal and distal ends, a longitudinal length and a lumen, an inner tubular member 30, 130 having opposed proximal and distal ends and a lumen, the intermediate tubular member 30, 130 slidably disposed within the outer tubular member lumen, an inner member 40, 140 slidably disposed within the intermediate tubular member 30, 130, and a plurality of elongate constrainment elements 50, 150 slidable within the intermediate tubular member, the constrainment elements 50, 150 having distal end structures moveable between a stent retaining configuration and a stent releasing configuration. A stent deployment region is provided on the inner member 40, 140 between the distal tip 42, 142 and the distal end 34, 134 of the intermediate tubular member 30, 130. A stent 60 with a suture 70 threaded through one end is placed over the inner member 40, 140 and between the distal tip 42, 142 and the distal end 34, 134 of the intermediate tubular member 30, 130. The suture 70 is removably held by the distal end structures on the constrainment elements 50, 150 that are in the stent retaining configuration.

The outer tubular member 20, 120 remains proximal of the stent, allowing the stent 60 to remain in the fully unconstrained configuration, as shown in FIGS. 1 and 5A. The stent 60 may be packaged in this configuration, with the stent 60 secured to the stent deployment device 10, 100. Prior to use, the stent 60 may be constrained within the device 10, 100. The outer tubular member 20, 120 may be axially moved distally over the stent 60, constraining the stent 60 into the deployment configuration as shown in FIG. 3. The stent 60 may be held within the device 10, 100 in the fully constrained configuration. The distal end structures of the constrainment elements 50, 150 retain the suture 70, thus maintaining the axial position of the proximal end of the stent 60 as the outer tubular member 20, 120 is advanced distally over the stent to radially compress the stent. The method may further include axially moving or sliding the outer tubular member 20, 120 toward a proximal position for releasing the stent 60. The method or use may yet further include providing an outer distal handle 22, 122 disposed at the proximal end of the outer tubular member 20, 120, providing an intermediate tubular handle 32, 132 disposed at the proximal end of the intermediate tubular member 30, 130, and providing a constrainment element handle 52, 152 disposed at the proximal end of the constrainment elements 50, 150, wherein independent axial movement of the outer tubular member 20, 120, the intermediate tubular member 30, 130 or the constrainment elements 50, 150 is achieved by manual manipulation of the handles 22, 122, 32, 132, and 52, 152.

The above method may be performed using a device 10 in which the distal end structures on the constrainment elements 50 are hooks 56 as shown in FIG. 2A, or a device 100 in which the distal end structures on the constrainment elements are straight distal ends of the wires, and the intermediate tubular member 130 has a recess 138 for receiving the suture 70 with the constrainment elements 50 holding the suture 70 in the recess 138, as discussed above and shown in FIGS. 5A and 5B. Other configurations of the constrainment elements 50 are also contemplated.

The materials that can be used for the various components of the delivery device 10, 100 (and/or other devices disclosed herein) and the various tubular members disclosed herein may include those commonly associated with medical devices. For simplicity purposes, the following discussion makes reference to outer tubular member 20 and intermediate tubular member 30 and other components of device 10. However, this is not intended to limit the devices and methods described herein, as the discussion may be applied to other similar devices and/or components of devices or devices disclosed herein.

The outer and intermediate tubular members 20, 30 may be formed of a body compatible material. Desirably, the biocompatible material is a biocompatible polymer. Examples of suitable biocompatible polymers include, but are not limited to, polyolefins such as polyethylene (PE), high density polyethylene (HDPE) and polypropylene (PP), polyolefin copolymers and terpolymers, polytetrafluoroethylene (PTFE), polyethylene terephthalate (PET), polyesters, polyamides, polyurethanes, polyurethaneureas, polypropylene and, polycarbonates, polyvinyl acetate, thermoplastic elastomers including polyether-polyester block copolymers and polyamide/polyether/polyesters elastomers, polyvinyl chloride, polystyrene, polyacrylate, polymethacrylate, polyacrylonitrile, polyacrylamide, silicone resins, combinations and copolymers thereof, and the like. Desirably, the biocompatible polymers include polypropylene (PP), polytetrafluoroethylene (PTFE), polyethylene terephthalate (PET), high density polyethylene (HDPE), combinations and copolymers thereof, and the like. The tubular members 20, 30 may be made of the same material or they may be made of different materials.

The outer and intermediate tubular members 20, 30 may also have a surface treatment and/or coating on their inner surface, outer surface or portions thereof. A coating need not be applied to both tubular members 20, 30, and individual members may be coated, uncoated, partially coated, and the like. Useful coating materials include any suitable biocompatible coating. Non-limiting examples of suitable coatings include polytetrafluoroethylene, silicone, hydrophilic materials, hydrogels, and the like. Useful hydrophilic coating materials include, but are not limited to, alkylene glycols, alkoxy polyalkylene glycols such as methoxypolyethylene oxide, polyoxyalkylene glycols such as polyethylene oxide, polyethylene oxide/polypropylene oxide copolymers, polyalkylene oxide-modified polydimethylsiloxanes, polyphosphazenes, poly(2-ethyl-2-oxazoline), homopolymers and copolymers of (meth) acrylic acid, poly(acrylic acid), copolymers of maleic anhydride including copolymers of methylvinyl ether and maleic acid, pyrrolidones including poly(vinylpyrrolidone) homopolymers and copolymers of vinyl pyrrolidone, poly(vinylsulfonic acid), acryl amides including poly(N-alkylacrylarnide), poly(vinyl alcohol), poly(ethyleneimine), polyamides, poly(carboxylic acids), methyl cellulose, carboxymethylcellulose, hydroxypropyl cellulose, polyvinylsulfonic acid, water soluble nylons, heparin, dextran, modified dextran, hydroxylated chitin, chondroitin sulphate, lecithin, hyaluranon, combinations and copolymers thereof, and the like. Non-limiting examples of suitable hydrogel coatings include polyethylene oxide and its copolymers, polyvinylpyrrolidone and its derivatives; hydroxyethylacrylates or hydroxyethyl(meth)acrylates; polyacrylic acids; polyacrylamides; polyethylene maleic anhydride, combinations and copolymers thereof, and the like. Additional details of suitable coating materials and methods of coating medical devices with the same may be found in U.S. Pat. Nos. 6,447,835 and 6,890,348, the contents of which are incorporated herein by reference. Such coatings and/or surface treatment is desirably disposed on the inside or a portion thereof of the outer tubular member 20 to aid, if desired, in loading and/or deploying of the stent 60.

Additionally, the various components of the devices/systems disclosed herein may include a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 320LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the delivery device 10 and/or other components of delivery system may be doped with, made of, or otherwise include a radiopaque material. Radiopaque materials are understood to be materials capable of producing a relatively bright image on a fluoroscopy screen or another imaging technique during a medical procedure. This relatively bright image aids the user of the delivery device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the delivery device 10 to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A stent delivery device comprising:
an outer tubular member having a distal end, a longitudinal length, and defining a lumen;
an intermediate tubular member having a distal end and defining a lumen, the intermediate tubular member disposed and slidable within the outer tubular member lumen; and
a plurality of elongate members slidable within one or more lumens of the intermediate tubular member, the plurality of elongate members having distal end structures moveable between a stent retaining configuration and a stent releasing configuration.

2. The stent delivery device of claim 1, wherein the distal end structures are in the stent retaining configuration when the distal end structures are extended distal of the distal end of the intermediate tubular member and in the stent releasing configuration when the distal end structures are retracted proximally within the intermediate tubular member.

3. The stent delivery device of claim 1, wherein the distal end structures are configured to retain a stent when the stent is in both a fully expanded and a constrained configuration.

4. The stent delivery device of claim 1, wherein the one or more lumens of the intermediate tubular member includes a plurality of channels extending longitudinally along the intermediate tubular member lumen, wherein each of the plurality of elongate member extends through a separate channel.

5. The stent delivery device of claim 4, wherein the distal end structures include hooks configured to retain a suture connected to a proximal end of a stent.

6. The stent delivery device of claim 5, wherein the hooks are configured to straighten and release the suture as the plurality of elongate members are withdrawn proximally into the intermediate tubular member channels.

7. The stent delivery device of claim 6, wherein the hooks have a width measured transverse to a longitudinal axis of the plurality of elongate members, wherein the width of the hooks is greater than a diameter of the intermediate tubular member channels.

8. The stent delivery device of claim 1, wherein the intermediate tubular member includes a recess in an outer wall thereof adjacent the distal end, wherein the plurality of elongate members are configured to pass through the recess and through a suture loop disposed within the recess when the plurality of elongate members are extended to the distal end of the intermediate tubular member in the stent retaining configuration, the plurality of elongate members configured to be retracted proximal of the recess to release the suture loop in the stent releasing configuration.

9. The stent delivery device of claim 8, wherein the recess extends circumferentially around an outer surface of the intermediate tubular member.

10. The stent delivery device of claim 1, wherein the outer tubular member, the intermediate tubular member, and the plurality of elongate members are all independently moveable relative to one another in a longitudinal direction.

11. The stent delivery device of claim 1, wherein the outer tubular member has an outer tubular member handle at a proximal end thereof, the intermediate tubular member has an intermediate tubular member handle at a proximal end thereof, and proximal ends of the plurality of elongate members are fixed to an actuation handle.

12. The stent delivery device of claim 11, wherein the actuation handle fixed to the plurality of elongate members is disposed between the outer tubular member handle and the intermediate tubular member handle.

13. A stent delivery device comprising:
an outer tubular member having a distal end, a longitudinal length, and defining a lumen;
an intermediate tubular member positioned in the outer tubular member lumen, the intermediate tubular member defining a lumen and having a distal end and a longitudinal length greater than the longitudinal length of the outer tubular member;
a plurality of elongate members extending through the intermediate tubular member lumen, the plurality of elongate members having proximal and distal ends, the distal ends configured to move between a stent retaining configuration and a stent releasing configuration;
the outer tubular member, the intermediate tubular member, and the plurality of elongate members being independently movable in a longitudinal direction;
the stent delivery device having a fully unconstrained loaded configuration, a fully constrained delivery configuration, and a stent deployment configuration;
wherein in both the fully unconstrained loaded and the fully constrained delivery configurations the distal ends of the plurality of elongate members are in the stent retaining configuration, positioned distal of the distal end of the intermediate tubular member; and
wherein in the stent deployment configuration the distal ends of the plurality of elongate members are in the stent releasing configuration, positioned proximal of the distal end of the intermediate tubular member.

14. The stent delivery device of claim 13, wherein the distal ends of the plurality of elongate members include hooks configured to retain a suture connected to a proximal end of a stent, the hooks configured to straighten as the plurality of elongate members are withdrawn proximally into the intermediate tubular member.

15. The stent delivery device of claim 13, wherein the intermediate tubular member includes a recess in an outer wall thereof adjacent the distal end, wherein the plurality of elongate members are configured to pass through the recess and through a suture loop disposed within the recess and connected to a proximal end of a stent when the plurality of elongate members are extended to the distal end of the intermediate tubular member in the stent retaining configuration, the plurality of elongate members configured to be retracted proximal of the recess to release the suture loop in the stent releasing configuration.

16. The stent delivery device of claim 13, wherein in the fully unconstrained loaded and stent delivery configurations, the distal end of the outer tubular member is positioned axially aligned with or proximal of the distal end of the intermediate tubular member, and in the fully constrained delivery configuration the distal end of the outer tubular member is positioned distal of the distal end of the intermediate tubular member.

17. A method for delivering a self-expanding stent into a bodily lumen, comprising:
radially contracting a stent on a stent delivery device, the stent delivery device comprising:
an outer tubular member having a distal end, a longitudinal length, and defining a lumen;
an intermediate tubular member having a distal end and defining a lumen, the intermediate tubular member disposed and slidable within the outer tubular member lumen;
an inner member slidable within the intermediate tubular member lumen;
a plurality of elongate members slidable within the intermediate tubular member, the plurality of elongate members having distal end structures moveable between a stent retaining configuration and a stent releasing configuration;
a self-expanding stent in an expanded configuration disposed over the inner member, a proximal end of the stent positioned distal of the distal ends of the intermediate and outer tubular members; and
a suture attached to the proximal end of the stent, the suture removably held by the distal end structures of the plurality of elongate members in the stent retaining configuration;
wherein radially contracting the stent onto the stent delivery device includes sliding the outer tubular member distally over the stent, thereby contracting the stent within the outer tubular member;
advancing the stent delivery device into the bodily lumen;
sliding the outer tubular member proximally until the distal end of the outer tubular member is proximal of the stent, thereby allowing the stent to expand; and
moving the distal end structures of the plurality of elongate members into the stent releasing configuration thereby releasing the stent.

18. The method of claim 17, wherein the distal end structures are in the stent retaining configuration when the distal end structures are extended distal of the distal end of the intermediate tubular member and moving the distal end structures into the stent releasing configuration includes retracting the distal end structures proximally within the intermediate tubular member.

19. The method of claim 18, wherein the distal end structures include hooks configured to retain the suture connected to the proximal end of the stent when the hooks are positioned distal of the distal end of the intermediate tubular member, the hooks configured to straighten and release the suture as the hooks are retracted proximally into the intermediate tubular member lumen.

20. The method of claim 19, wherein the intermediate tubular member includes a plurality of channels extending longitudinally along the intermediate tubular member, wherein each of the plurality of elongate member extends through a separate channel, wherein the hooks have a width measured transverse to a longitudinal axis of the plurality of elongate members, wherein the width of the hooks is greater than a diameter of the intermediate tubular member channels.
